(19) United States
(12) Patent Application Publication
(10) Pub. No.: US 2024/0159776 A1
(43) Pub. Date: May 16, 2024

(54) Precision Medicine Approach to Targeting Neurodegeneration

(71) Applicant: UNIVERSITY OF NORTH TEXAS HEALTH SCIENCE CENTER AT FORT WORTH, Fort Worth, TX (US)

(72) Inventor: O'Bryant Sid E., Aledo, TX (US)

(21) Appl. No.: 18/549,183

(22) PCT Filed: Mar. 7, 2022
(86) PCT No.: PCT/US2022/019101

§ 371 Date: Sep. 6, 2023

Related U.S. Application Data
(60) Provisional application No. 63/159,812, filed on Mar. 11, 2021.

(51) Int. Cl. G01N 33/68; A61K 45/06; G01N 33/92

(52) U.S. Cl. CPC G01N 33/6896; A61K 45/06; G01N 33/6869; G01N 33/92; G01N 2333/525; G01N 2333/5409; G01N 2333/5412; G01N 2333/5418; G01N 2333/5428; G01N 2333/605; G01N 2333/62; G01N 2333/745; G01N 2800/2814; G01N 2800/56

(57) ABSTRACT

The present invention includes methods for detecting neurodegeneration and treating a subject that is of Mexican American or non-Hispanic white origin, the method comprising: obtaining a blood, plasma or serum sample; determining ethnicity of the subject; measuring one or more biochemical biomarkers; or measuring one or more protein biomarkers, or measuring both biochemical biomarkers and protein biomarkers; comparing the level of expression from the sample with a statistical sample representative of the subject of Mexican American or of non-Hispanic white origin, suspected of having neurodegeneration; and treating the subject with a treatment that targets neurodegeneration or neuronal injury, wherein the neurodegeneration or neuronal injury is measured by [18F]-fluorodeoxyglucose-PET, structural MRI, or CSF total tau.

### STATEMENT OF FEDERALLY FUNDED RESEARCH

This invention was made with government support under AG054073 and AG058533 awarded by the National Institutes of Health. The government has certain rights in the invention.

### TECHNICAL FIELD OF THE INVENTION

The present invention relates in general to the field of precision medicine approach to targeting neurodegeneration.

### INCORPORATION-BY-REFERENCE OF MATERIALS FILED ON COMPACT DISC

Not applicable.

### BACKGROUND OF THE INVENTION

Without limiting the scope of the invention, its background is described in connection with neurodegenerative pathology.

Neurodegeneration is a pathology of complex etiology where neuronal tissue or cells is compromised, damaged or deteriorated. Neurodegeneration can result from injury, accident, disease and the aging process. Neurodegeneration can occur in subjects of all ages and ethnicities. Neurodegeneration is currently detected in a subject by the use of complex, expensive and time-consuming methods, including [18F]-fluorodeoxyglucose—PET, structural MRI, or CSF total tau.

Despite the prevalence of these methods, there is a significant need for detection of neurodegeneration by methods that are simpler, require less time and are not as expensive as current methods.

### SUMMARY OF THE INVENTION

In one embodiment, the present invention includes a method for detecting neurodegeneration and treating a subject that is of Mexican American or non-Hispanic white origin, the method comprising: obtaining a blood, plasma or serum sample from the subject comprising both biochemical and protein biomarkers; determining if the subject is of Mexican American or of non-Hispanic white origin; measuring in the blood, plasma or serum sample a level of one or more biochemical biomarkers selected from the group consisting of: cholesterol, triglycerides, HDL cholesterol, glucose, HBAc1, LDL cholesterol, and glucagon; or measuring in the blood, plasma or serum sample an expression level of one or more protein biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10, or both biochemical biomarkers and protein biomarkers; comparing the level of expression from the sample with a statistical sample representative of the subject of Mexican American or of non-Hispanic white origin, suspected of having neurodegeneration; and treating the subject with a treatment that targets neurodegeneration or neuronal injury, wherein the neurodegeneration or neuronal injury is measured by [18F]-fluorodeoxyglucose-PET, structural MRI, or CSF total tau. In one aspect, the subject is of Mexican American origin and suffering neurodegeneration with or without mild cognitive impairment or dementia. In another aspect, the subject is of Mexican American origin, neurodegeneration is screened for by detecting the level of biochemical biomarkers and the level of expression of biomarkers, selected from NFL, A2M, Ab40, B2M, FABP3, HBA1c, HDL cholesterol, IL-6, Ab42, IL-5, TNC, eotaxin-3, PPY, tau, glucose, triglycerides, SAA, TNFα, cholesterol, IL-7, TPO, sICAM-1, IL-18, Factor VII, sVCAM-1, LDL cholesterol, CRP, GLP-1, insulin, IL-10, TARC, PYY, glucagon, and I309, and optionally, wherein the biochemical and protein biomarkers are in descending order. In another aspect, if the subject is of Mexican American origin and suffering from mild cognitive impairment, neurodegeneration is screened for by detecting the level of biochemical biomarkers and the level of expression of biomarkers, selected from NFL, glucose, HBAc1, 1309, PYY, sVCAM-1, B2M, triglycerides, TNFα, PPY, eotaxin-3, cholesterol, Ab40, GLP-1, IL-10, SAA, IL-5, and TPO, and optionally, wherein the biochemical and protein biomarkers are in descending order. In another aspect, if the subject is of Mexican American origin and suffering from dementia, neurodegeneration is screened for by detecting the level of biochemical biomarkers and the level of expression of biomarkers, selected from TNFα, eotaxin-3, NFL, IL-10, FABP3, IL-6, cholesterol, I309, sVCAM-1, PPY, sICAM-1, TPO, factor VII, Ab40, IL-18, Ab42, HDL cholesterol, SAA, A2M, LDL cholesterol, and GLP-1, and optionally, wherein the biochemical and protein biomarkers are in descending order. In another aspect, the subject is of non-Hispanic white origin and suffering from neurodegeneration with or without mild cognitive impairment or dementia. In another aspect, if the subject is of non-Hispanic white origin neurodegeneration is screened for by detecting the level of biochemical biomarkers and the level of expression of biomarkers, selected from cholesterol, A2M, Factor VII, TNC, HDL cholesterol, IL-18, Ab42, NFL, Ab40, LDL cholesterol, TARC, IL-6, TNFα, B2M, IL-5, TPO, I309, triglycerides, SAA, insulin, PPY, tau, GLP-1, glucose, FABP3, sICAM-1, sVCAM-1, PYY, eotaxin-3, IL-7, CRP, IL-10, HBA1c, and glucagon, and optionally, wherein the biochemical and protein biomarkers are in descending order. In another aspect, if the subject is of non-Hispanic white origin and suffering from mild cognitive impairment, neurodegeneration is screened for by detecting the level of biochemical biomarkers and the level of expression of biomarkers, selected from triglycerides, HDL cholesterol, glucose, HBAc1, A2M, GLP-1, IL-7, IL-6, PYY, IL-10, glucagon, Ab40, FABP3, Ab42, eotaxin-3, and Factor VII, and optionally, wherein the biochemical and protein biomarkers are in descending order. In another aspect, if the subject is of non-Hispanic white origin and suffering from dementia, neurodegeneration is screened for by detecting the level of biochemical biomarkers and the level of expression of biomarkers, selected from HDL cholesterol, Ab42, A2M, FABP3, TNC, TARC, glucose, TPO, CRP, and eotaxin-3, and optionally, wherein the biochemical and protein biomarkers are in descending order. In another aspect, the method further comprises wherein: (i) when the expression level of the one or more biomarkers in the blood, plasma or serum sample is statistically similar to the average expression level of the corresponding one or more biomarkers obtained from a group of individuals in the statistical sample who do not have neurodegeneration, the subject is excluded from further testing for neurodegeneration; or (ii) when the expression level of the one or more biomarkers in the blood, plasma or serum sample is not statistically similar to the average expression level of the corresponding one or more biomarkers obtained from a group of individuals in the statistical sample who have been diagnosed with neurodegeneration, the subject is further tested for neurodegeneration. In another aspect, the method further testing for neurodegeneration excluded is selected from MRI, PET, or spinal fluid tap. In another aspect, the expression level of the one or more biomarkers in the blood, plasma or serum sample is statistically similar to the average expression level of the corresponding one or more biomarkers obtained from a group of individuals in the statistical sample who have been diagnosed with neurodegeneration, the method further comprises referring the subject to a specialist for diagnostic testing for a neurological disease or disorder.

In another embodiment, the present invention includes a method of screening a subject for exclusion from a clinical trial for a neuroprotection agent, the method comprising: obtaining a blood, plasma or serum sample from the subject comprising both biochemical and protein biomarkers; determining if the subject is of Mexican American or of non-Hispanic white origin; measuring in the blood, plasma or serum sample a level of one or more biochemical biomarkers selected from the group consisting of: cholesterol, triglycerides, HDL cholesterol, glucose, HBAc1, LDL cholesterol, and glucagon; or measuring in the blood, plasma or serum sample an expression level of one or more protein biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10, or both biochemical and protein biomarkers; comparing the level of expression from the sample with a statistical sample representative of the subject of Mexican American or of non-Hispanic white origin, suspected of having neurodegeneration; and determining that the subject has a neurodegeneration profile based on the expression level of the one or more biomarkers; and excluding the subject for inclusion in the clinical trial targeting neuroprotection if the subject has the neurodegeneration profile. In one aspect, if the subject is of Mexican American origin, the neurodegeneration profile is screened for by detecting the level of biochemical biomarkers and the level of expression of biomarkers, selected from: NFL, A2M, Ab40, B2M, FABP3, HBA1c, HDL cholesterol, IL-6, Ab42, IL-5, TNC, eotaxin-3, PPY, tau, glucose, triglycerides, SAA, TNFα, cholesterol, IL-7, TPO, sICAM-1, IL-18, Factor VII, sVCAM-1, LDL cholesterol, CRP, GLP-1, insulin, IL-10, TARC, PYY, glucagon, and I309, and optionally, wherein the biochemical and protein biomarkers are in descending order. In another aspect, if the subject is of Mexican American origin and suffering from mild cognitive impairment, the neurodegeneration profile is screened for by detecting the level of biochemical biomarkers and the level of expression of biomarkers, selected from NFL, glucose, HBAc1, 1309, PYY, sVCAM-1, B2M, triglycerides, TNFα, eotaxin-3, cholesterol, Ab40, GLP-1, IL-10, SAA, IL-5, and TPO, and optionally, wherein the biochemical and protein biomarkers are in descending order. In another aspect, if the subject is of Mexican American origin and suffering from dementia, the neurodegeneration profile is screened for by detecting the level of biochemical biomarkers and the level of expression of biomarkers, selected from TNFα, eotaxin-3, NFL, IL-10, FABP3, IL-6, cholesterol, I309, sVCAM-1, PPY, sICAM-1, TPO, factor VII, Ab40, IL-18, Ab42, HDL cholesterol, SAA, A2M, LDL cholesterol, and GLP-1, and optionally, wherein the biochemical and protein biomarkers are in descending order. In another aspect, if the subject is of non-Hispanic white origin the neurodegeneration profile is screened for by detecting the level of biochemical biomarkers and the level of expression of biomarkers, selected from cholesterol, A2M, Factor VII, TNC, HDL cholesterol, IL-18, Ab42, NFL, Ab40, LDL cholesterol, TARC, IL-6, TNFα, B2M, IL-5, TPO, I309, triglycerides, SAA, insulin, PPY, tau, GLP-1, glucose, FABP3, sICAM-1, sVCAM-1, PYY, eotaxin-3, IL-7, CRP, IL-10, HBA1c, and glucagon, and optionally, wherein the biochemical and protein biomarkers are in descending order. In another aspect, if the subject is of non-Hispanic white origin and suffering from mild cognitive impairment, the neurodegeneration profile is screened for by detecting the level of biochemical biomarkers and the level of expression of biomarkers, selected from triglycerides, HDL cholesterol, glucose, HBAc1, A2M, GLP-1, IL-7, IL-6, PYY, IL-10, glucagon, Ab40, FABP3, Ab42, eotaxin-3, and Factor VII, and optionally, wherein the biochemical and protein biomarkers are in descending order. In another aspect, if the subject is of non-Hispanic white origin and suffering from dementia, the neurodegeneration profile is screened for by detecting the level of biochemical biomarkers and the level of expression of biomarkers, selected from HDL cholesterol, Ab42, A2M, FABP3, TNC, TARC, glucose, TPO, CRP, and eotaxin-3, and optionally, wherein the biochemical and protein biomarkers are in descending order.

In another embodiment, the present invention includes a method for detecting neurodegeneration in a subject that is of Mexican American origin, the method comprising: (a) obtaining or having obtained a blood, plasma or serum sample from the subject comprising both biochemical and protein biomarkers; (b) determining if the subject has diabetes and a duration of the diabetes; (c) measuring in the blood, plasma or serum sample one or more biochemical biomarkers of diabetes selected from HbA1c, glucose; or (d) measuring in the blood, plasma or serum sample an expression level of one or more biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10; (e) comparing the level of expression from the sample with a statistical sample representative of the subject of Mexican American or of non-Hispanic white origin, suspected of having a neurodegenerative disease; and (f) treating the subject with a treatment that targets neurodegeneration or neuronal injury, wherein the neurodegeneration or neuronal injury is measured by [18F]-fluorodeoxyglucose-PET, structural MRI, or CSF total tau. In another aspect, the method further comprises at least one of: determining if the subject has neurodegeneration by detecting the level of expression of biomarkers, selected from NFL, A2M, Ab40, B2M, FABP3, IL-6, Ab42, IL-5, TNC, eotaxin-3, PYY, PPY, tau, SAA, TNFα, cholesterol, IL-7, TPO, sICAM-1, IL-18, Factor VII, sVCAM-1, CRP, GLP-1, insulin, IL-10, TARC, and I309, and optionally, wherein the biomarkers are in descending order;

In another embodiment, the present invention includes a method for detecting neurodegeneration in a subject of non-Hispanic white origin, the method comprising: (a) obtaining a blood, plasma or serum sample from the subject comprising both biochemical and protein biomarkers; (b) determining if the subject has a cardiovascular disease and hypertension; (c) measuring in the blood, plasma or serum sample a level of one or more biochemical biomarkers selected from LDL, and total cholesterol; or (d) measuring in the blood, plasma or serum sample an expression level of one or more biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10; (e) comparing the level of expression from the sample with a statistical sample representative of the subject of Mexican American or of non-Hispanic white origin, suspected of having a neurodegenerative disease; and (f) treating the subject with a treatment that targets neurodegeneration or neuronal injury, wherein the neurodegeneration or neuronal injury is measured by [18F]-fluorodeoxyglucose-PET, structural MRI, or CSF total tau. In one aspect, the method further comprises determining if the subject has a neurodegenerative disease by determining the level of expression of biomarkers selected from A2M, Factor VII, TNC, IL-18, Ab42, NFL, Ab40, TARC, IL-6, TNFα, B2M, IL-5, TPO, I309, triglycerides, SAA, insulin, PYY, PPY, tau, GLP-1, FABP3, sICAM-1, sVCAM-1, eotaxin-3, IL-7, CRP, and IL-10, and optionally, wherein the biomarkers are in descending order.

### BRIEF DESCRIPTION OF THE DRAWINGS

For a more complete understanding of the features and advantages of the present invention, reference is now made to the detailed description of the invention along with the accompanying figure(s) and in which:

FIG. 1 shows the proteomic profile of neurodegeneration in HABLE cohort.

FIGS. 2A and 2B show proteomic profile of neurodegeneration by ethnicity. FIG. 2A shows the proteomic profile of neurodegeneration among non-Hispanic whites (NHW). FIG. 2B shows the proteomic profile of neurodegeneration among Mexican Americans (MA).

FIGS. 3A to 3F show the proteomic profile of neurodegeneration by ethnicity and diagnostic status. FIG. 3A: NHW Controls, FIG. 3B: NHW mild cognitive impairment (MCI), FIG. 3C: NHW Dementia, FIG. 3D: MA Control, FIG. 3E: MA MCI, FIG. 3F: MA Dementia.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the phrase “neurological disease” refers to a disease or disorder of the central nervous system and many include, e.g., disorders such as Alzheimer's Disease, Parkinson's disease, mild cognitive impairment (MCI) and dementia and neurological diseases include multiple sclerosis and neuropathies.

As used herein, the term “neurodegeneration” refers to a pathology of complex etiology where neuronal tissue or cells is compromised, damaged or deteriorated. Neurodegeneration includes any pathological state that results in the progressive loss of neural cell or tissue structures or function, including neural cell death. Neurodegeneration is a pathological state caused by neurological disorders.

As used herein, the terms “Alzheimer's patient”, “AD patient”, and “individual diagnosed with AD” all refer to an individual who has been diagnosed with AD or has been given a probable diagnosis of Alzheimer's Disease (AD).

As used herein, the term “biomarker”, refers to any of: a protein biomarkers or substances that are functionally at the level of a protein biomarker.

As used herein, the terms “cognition”, “cognitive ability”, “memory”, “language” and the like are used interchangeably to refer to an individual's ability to perform cognitive abilities and the dysfunction of those abilities that may be as a result of a diagnosis of a neurological disease as well as other medical and psychiatric conditions including, but not limited to, diabetes, hypertension, dyslipidemia, metabolic syndrome, depression, traumatic brain injury, schizophrenia, bipolar disease, as well as the cognitive slowing/decline associated with the aging process itself.

As used herein, “biological fluid sample” refers to a wide variety of fluid sample types obtained from an individual and can be used in a diagnostic or monitoring assay. Biological fluid sample include, e.g., blood, plasma, serum, cerebral spinal fluid (CSF), urine and other liquid samples of biological origin. Commonly, the samples are treatment with stabilizing reagents, solubilization, or enrichment for certain components, such as proteins or polynucleotides, so long as they do not interfere with the analysis of the markers in the sample.

As used herein, a “blood sample” refers to a biological sample derived from blood, preferably peripheral (or circulating) blood. A blood sample may be, e.g., whole blood, serum or plasma. In certain embodiments, serum is preferred as the source for the biomarkers as the samples are readily available and often obtained for other sampling, is stable, and requires less processing, thus making it ideal for locations with little to refrigeration or electricity, is easily transportable, and is commonly handled by medical support staff.

As used herein, a “normal” individual or a sample from a “normal” individual refers to quantitative data, qualitative data, or both from an individual who has or would be assessed by a physician as not having a disease, e.g., a neurological disease. Often, a “normal” individual is also age-matched within a range of 1, 2, 3, 4, 5, 6, 7, 8, 9 or 10 years with the sample of the individual to be assessed.

As used herein, the term “treatment” refers to the alleviation, amelioration, and/or stabilization of symptoms, as well as delay in progression of symptoms of a particular disorder. For example, “treatment” of AD includes any one or more of: (1) elimination of one or more symptoms of AD, (2) reduction of one or more symptoms of AD, (4) stabilization of the symptoms of AD (e.g., failure to progress to more advanced stages of AD), and (5) delay in onset of one or more symptoms of AD delay in progression (i.e., worsening) of one or more symptoms of AD; and (6) delay in progression (i.e., worsening) of one or more symptoms of AD.

As used herein, intracellular adhesion molecule-1 (ICAM1 or ICAM-1) is interchangeable with soluble intracellular adhesion molecule-1 (sICAM1 or sICAM-1).

As used herein, vascular cell adhesion molecule-1 (VCAM1 or VCAM-1) is interchangeable with soluble vascular cell adhesion molecule-1 (sVCAM1 or sVCAM-1).

As used herein, FABP and FABP3 are used interchangeably to refer to fatty acid binding protein.

As used herein a “statistical sample representative of the subject” or a “statistical sample representative of the patient” refers to a statistical sample comprising one or more of the following groups of individuals: (1) individuals suspected of having neurodegeneration; (2) individuals not suspected of having neurodegeneration; (3) individuals suspected of having neurodegeneration with or without mild cognitive impairment or dementia; and (4) individuals not suspected of having neurodegeneration with or without mild cognitive impairment or dementia.

In some embodiments, the data obtained from the patient or subject (e.g., demographic factors, neurocognitive evaluation results, and biomarker expression level) are compared to the corresponding data from individuals in the statistical sample. This comparison is discussed elsewhere herein and can be applied to any method of the present disclosure.

The present inventors examined proteomic profiles of a MRI-based marker of neurodegeneration from the AT(N) framework among a multi-ethnic, community-dwelling cohort. Community-dwelling Mexican Americans and non-Hispanic white adults and elders were recruited. All participants underwent comprehensive assessments including an interview, functional exam, clinical labs, informant interview, neuropsychological testing and 3T MRI of the brain. A Neurodegeneration MRI meta ROI biomarker for the AT(N) framework was calculated. The data was examined from n=1,291 participants. Proteomic profiles were highly accurate for detecting neurodegeneration (i.e., N+) among both Mexican Americans and non-Hispanic whites. It was found that the proteomic profile of N+ was different between ethnic groups. Further analyses revealed that the proteomic profiles of N+ varied by diagnostic status (control, MCI, dementia) and ethnicity (Mexican American vs. non-Hispanic whites) though diagnostic accuracy was high for all classifications.

The present invention is a biochemical and proteomic profile of neurodegeneration for novel diagnoses and intervention. It is also shown herein that the underlying biological factors associated with neurodegeneration are different between Mexican Americans and non-Hispanic whites as well as at different levels of disease progression.

The present invention includes detecting neurodegeneration and treating a subject that is of Mexican American or non-Hispanic white origin. First, whether a blood-based profile could predict N+ was determined. The context of use (COU) of this blood test is as follows: a blood screening test to rule out neurodegeneration among patients being considered for a clinical trial targeting neuroprotection. This specific COU has the advantage of being cost-effective, scalable, rapid and could serve as the first step in the trial screening process with those who screen positive undergoing structural MRI for confirmation of N+. Second, the inventors determined whether a profile of N+ varied by ethnicity and by disease severity. It was found that biological factors associated with MCI and AD vary between Mexican Americans and non-Hispanic whites. For example, when compared to non-Hispanic whites, amyloid positivity rates are lower, blood-based profiles of MCI and dementia are significantly different, DTI-based profiles of MCI and dementia are significantly different, and even APOE4 genotype prevalence rates are different. In addition, the inventors have found that medical comorbidities and sociocultural factors that are disproportionately suffered by Mexican Americans are also differentially linked to these biological markers. Therefore, the diversity of medical comorbidities may be an ideal situation to begin studying a precision medicine approach by identifying subgroups for targeted interventions. Given the recent literature supporting plasma NfL as a putative biomarker of neurodegeneration, the inventors conducted a comparison analyses with this marker alone to determine if it, by itself, could serve the COU rather than a broader profile approach.

Participants & Assessment. The HABLE study is an ongoing, longitudinal, community-based project examining health disparities in MCI and AD among Hispanic, Mexican Americans. Detailed HABLE recruitment methods are being published elsewhere. Baseline visits of the HABLE study will continue until n=1,000 per group are enrolled. Recruiting has been slowed due to COVID-19. The HABLE protocol includes an interview (containing questions pertaining to SES, acculturation, social support, chronic stress, and depression), functional exam, blood draw for clinical labs and biobanking, neuropsychological testing and 3T MRI of the brain. All aspects of the study protocol can be conducted in Spanish or English. The HABLE study is conducted under IRB approved protocols and each participant (or his/her legal representative) signs written informed consent. The neuropsychological test battery includes the following: Mini Mental Status Exam (MMSE)[12], Wechsler Memory Scale—Third Edition (WMS-III) Digit Span and Logical Memory [13], Digit Symbol Substitution, Trail Making Test Parts A and B[14], Spanish-English Verbal Learning Test (SEVLT)[15], Animal Naming (semantic fluency)[16], FAS (phonemic fluency)[16] as well as the American National Adult Reading Test (English-speakers)[17] and Word Accentuation Test (Spanish-speakers)[18]. An informant interview is also conducted for completion of the Clinical Dementia Rating Scale[19] by clinicians with expertise in dementia.

Diagnostic Classification. Cognitive diagnoses are assigned algorithmically (decision tree) and verified at consensus review as follows: Normal Control (NC)=no cognitive complaints, CDR sum of boxes score of 0 and cognitive tests scores broadly within normal limits; Mild Cognitive Impairment (MCI): cognitive complaint (self or other), CDR sum of boxes score between 0.5-2.0 and at least one cognitive test score falling 1.5 standard deviation below normative ranges; Dementia: CDR sum of boxes score >=2.5 and at least two cognitive test scores 2 standard deviation below normative ranges.

Neuroimaging. MRI Data. The HABLE MRI protocol is based on that of ADNI3 using a 3T Siemens Magnetom SKYRA whole-body scanner. The inventors acquired the following scan sequences: T1-weighted whole brain volumetric spoiled Magnetization-Prepared Rapid Gradient (MPRAGE), whole brain volumetric fluid attenuated inversion recovery (FLAIR), susceptibility-weighted imaging (SWI), diffusion tensor MRI (dMRI), 3D arterial spin labeling (3DPASL), resting-state functional (rsfMRI), and high resolution (0.4×0.4 mm×2 mm) T2-weighted hippocampal high resolution (HHR) scans. For this study, the “meta-ROI” for examination of the neurodegeneration (i.e., N) component of the AT(N) framework[1] was examined as outlined by Jack et al[4].

Blood Collection & Processing Procedures. Fasting blood collection and processing were completed based on the international guidelines for AD biomarker studies and processed within 2 hours (stick-to-freezer) [20]. Plasma NfL, Aβ40, A1342, total tau (t-tau) and NfL were assayed using the ultra-sensitive Simoa (single molecule array) technology platform HD-1 (Quanterix.com). The ITR Biomarker Core has conducted >5,000 NfL assays using this platform and all coefficients of variability (CVs) were <4%. Plasma ECL markers were assayed per previously published protocols [21-24]. Assay preparation was completed with the Hamilton Robotics StarPlus system. The ITR Biomarker Core has conducted >20,000 assays using this platform and all coefficients of variability (CVs) were <=10%. Clinical labs were conducted by a local Quest laboratory per HABLE protocols.

Statistical Analyses. Statistical Analyses were conducted in SPSS 25 (IBM) and R [25]. Independent t-tests and Mann Whitney U tests were conducted to examine differences in demographic characteristics for categorical. Statistical analyses were conducted using the R (V 3.3.3) statistical software, SPSS 24 (IBM) and SAS. Independent t-tests were applied to examine differences in demographic characteristics. Support vector machine (SVM) analyses were also conducted. SVM is based on the concept of decision planes that define decision boundaries and is primarily a classifier method that performs classification tasks by constructing hyperplanes in a multidimensional space that separates cases of different class labels. A 10 times repeated 5-fold cross-validation was used. Ten times repeated 5-fold cross-validation is used to directly perform SVM parameter tuning and optimal cutoff determination using Grid Search which is traditional way of performing hyperparameter optimization [26]. In 5-fold cross-validation, the data is divided into 5 folds. The model is trained on 4 folds with one-fold held back for testing. This process gets repeated to ensure each fold of the dataset gets the chance to be the held back set. 10 times repeated 5-fold cross-validation repeats the process of 5-fold cross-validation 10 times. Once the process is completed, the evaluation metrics are summarized using the mean. The advantage of 10 times repeated 5-fold cross-validation is that it can provide a more reliable estimate of out-of-sample performance by reducing the variance associated with a single trial of cross-validation. Diagnostic accuracy was calculated via receiver operating characteristic (ROC) curves. Sensitivity (SN), specificity (SP), negative predictive value (NPV, the probability of not having the condition of interest based on a negative test result) and positive predictive value (PPV, the probability of having the condition of interest based on a positive test) statistics were calculated. Analyses were conducted as follows: (1) detecting N+ versus N− in the entire cohort, (2) detecting N+ versus N− split by ethnicity, and (3) detecting N+ versus N− split by ethnicity and diagnostic status.

Of the total n=1,761 actively enrolled participants, a total of n=1,291 participants had all requisite data (i.e., passed all QA for all bilateral reference regions of interest for calculation of the weighted meta-ROI, had proteomics data) for inclusion in the current analyses (Mexican American n=607, non-Hispanic white n=684). The Mexican American group was significantly younger (p<0.001) and obtained fewer years of education (p<0.001) than non-Hispanic whites. Mexican Americans were more likely to have a diagnosis of hypertension (p<0.05) and diabetes (p<0.001) than non-Hispanic whites. Demographic characteristics of the cohort, by ethnic group, is presented in Table 1.

TABLE 1
Descriptive Statistics of Cohort (n = 1,291)

| | | Mexican American | Non-Hispanic White |
|---|---|---|---|
| | Total Cohort | N = 684 | N = 607 |
| Age | 66.02 (8.65) | 63.57 (7.91) | 68.76 (8.63) |
| Education | 12.37 (4.73) | 9.63 (4.58) | 15.44 (2.47) |
| Gender (% female) | 64% | 70% | 58% |
| Hypertension | 58% | 61% | 55% |
| (% yes) | | | |
| Diabetes (% yes) | 25% | 36% | 12% |
| Dyslipidemia | 62% | 64% | 60% |
| (% yes) | | | |
| MRI N | 2.72 (0.15) | 2.73 (0.14) | 2.71 (0.15) |
| Normal Controls | 81% (n = 1050) | 78% (n = 532) | 85% (n = 518) |
| MCI | 13% (n = 168) | 16% (n = 107) | 10% (n = 61) |
| Dementia | 6% (n = 73) | 7% (n = 45) | 5% (n = 28) |

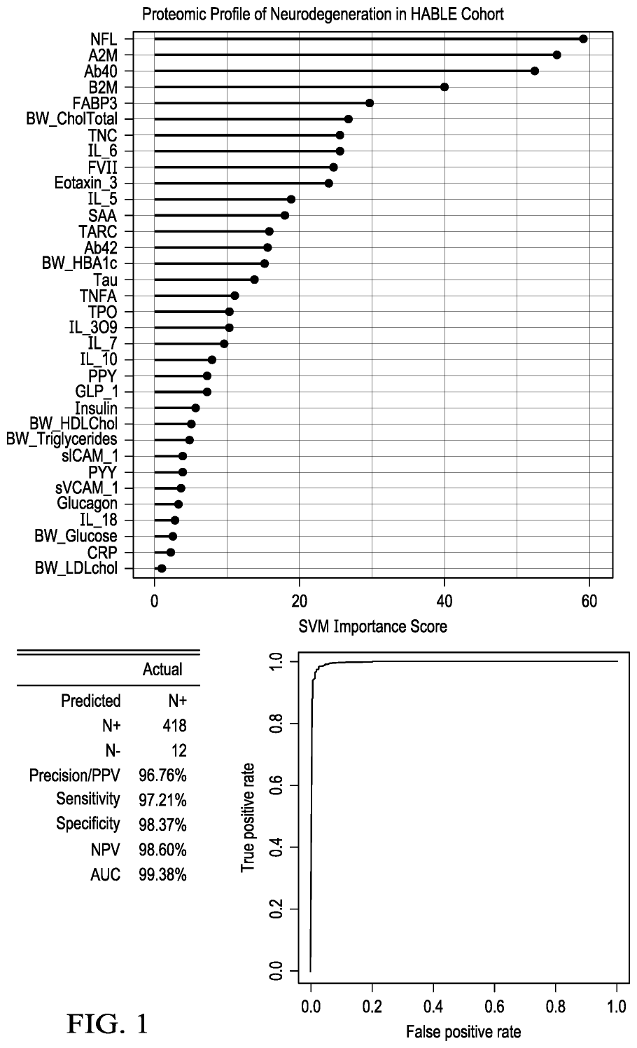

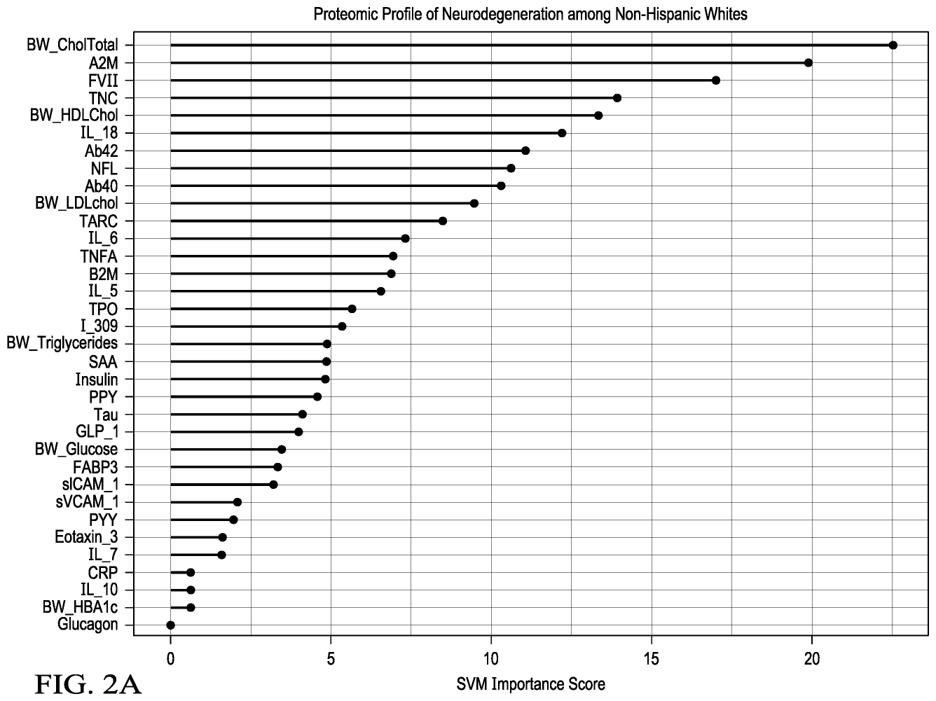

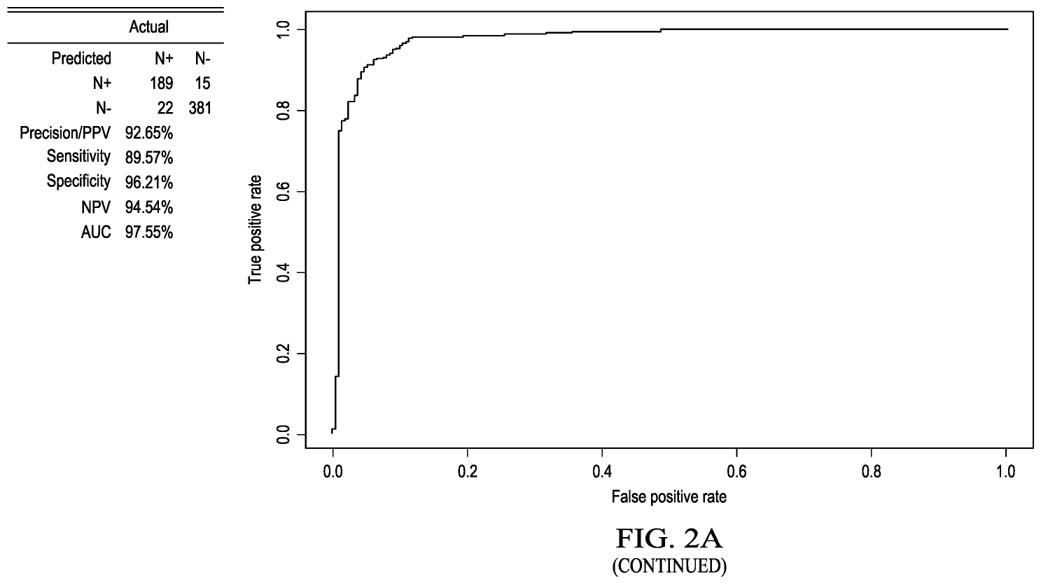

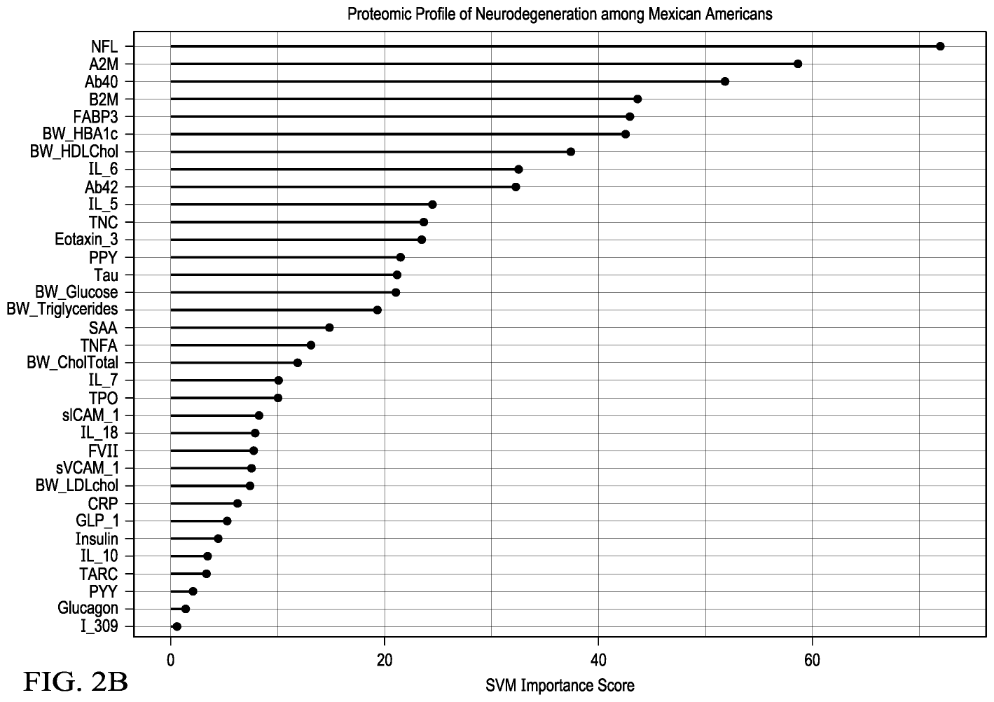

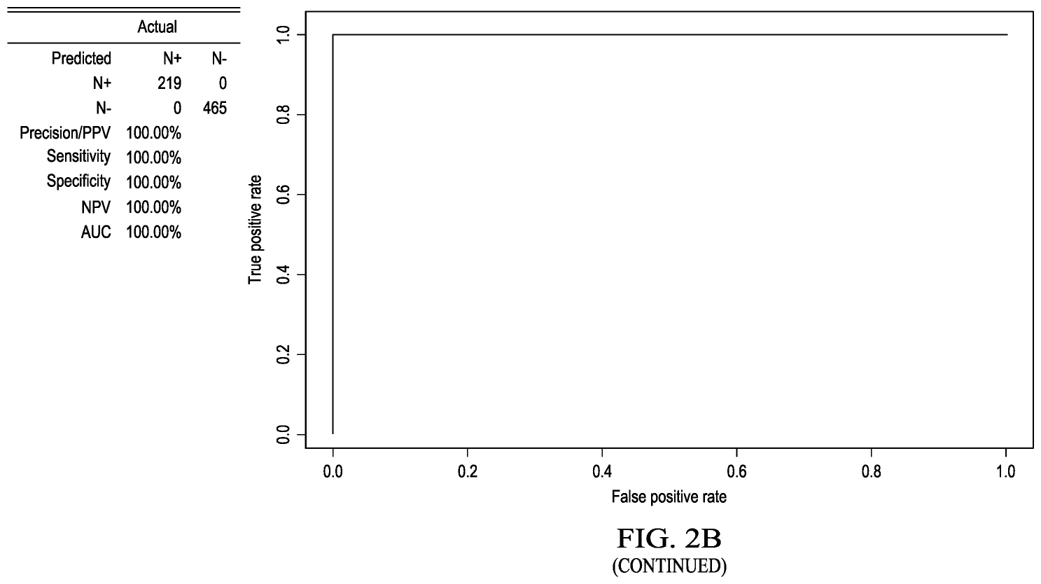

Blood Profile of N+ Within Entire Cohort and Split By Ethnicity. The SVM blood-based profile of neurodegeneration in the entire cohort was highly accurate, AUC=0.99, SN=0.97, SP=0.98, NPV=99%, PPV=96% (FIG. 1). For comparison purposes, plasma NfL alone yielded an AUC=0.67. When split by ethnicity, the blood-based profile was highly accurate in detecting N+ among non-Hispanic whites (AUC=0.98, SN=0.90, SP=0.96, PPV=93%, and NPV=95% (FIG. 2A) as well as Mexican Americans (AUC=1.0, SN=1.0, SP=1.0, PPV=100%, NPV=100%) (FIG. 2B). While all profiles were highly accurate, the relative importance of the markers in the profile varied substantially between ethnic groups. For comparison purposes, plasma NfL yielded an AUC=0.64 among non-Hispanic whites and 0.68 among Mexican Americans.

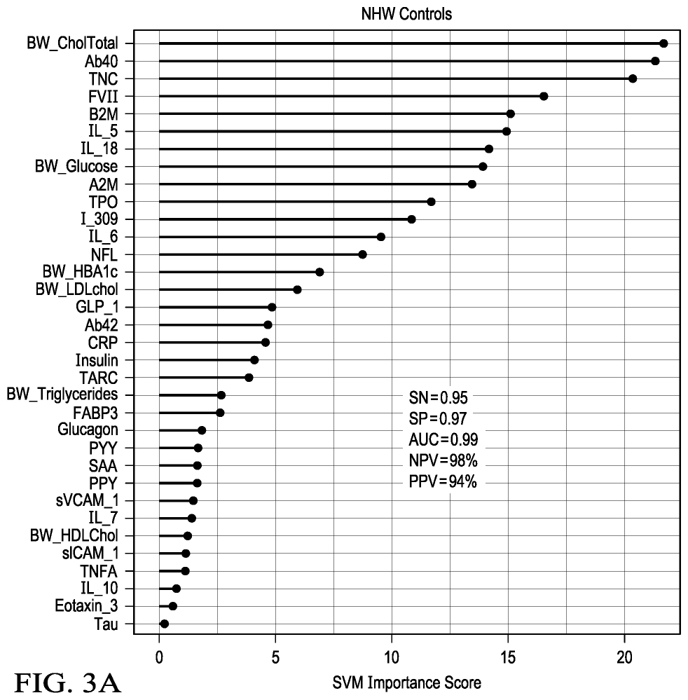

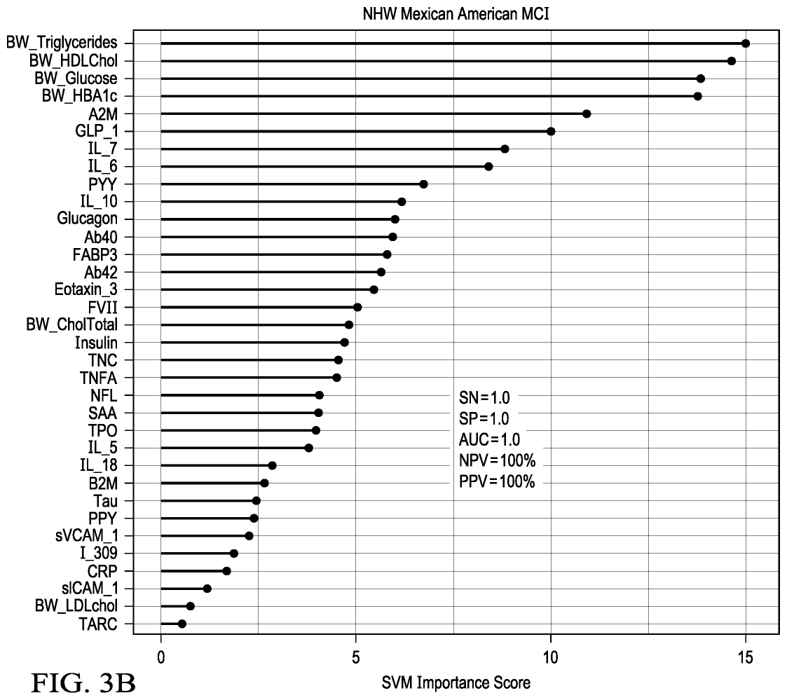

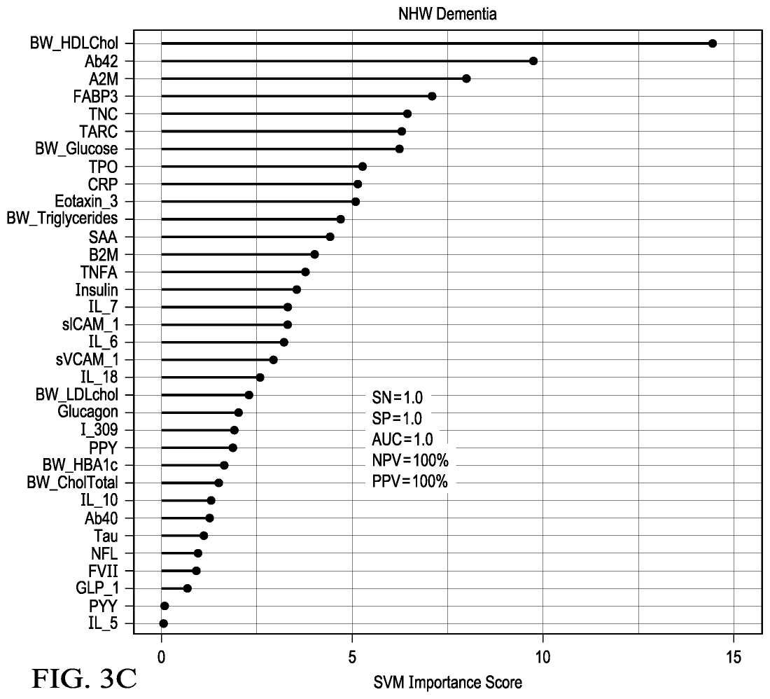

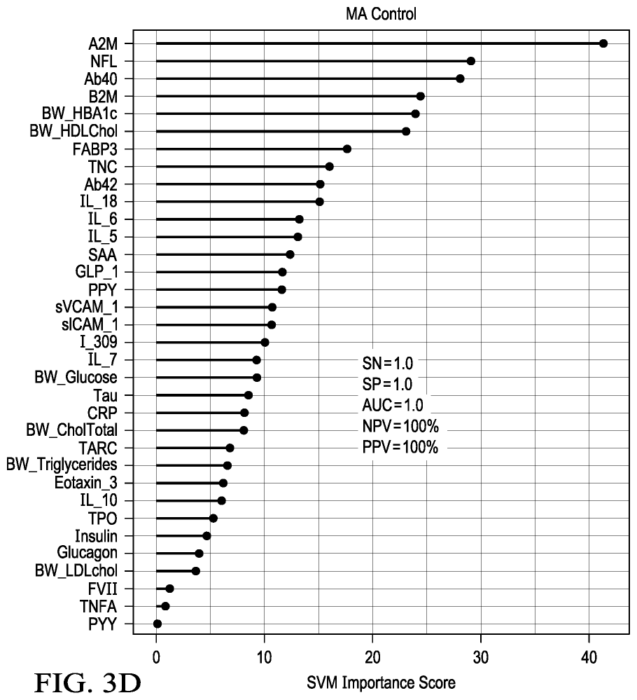

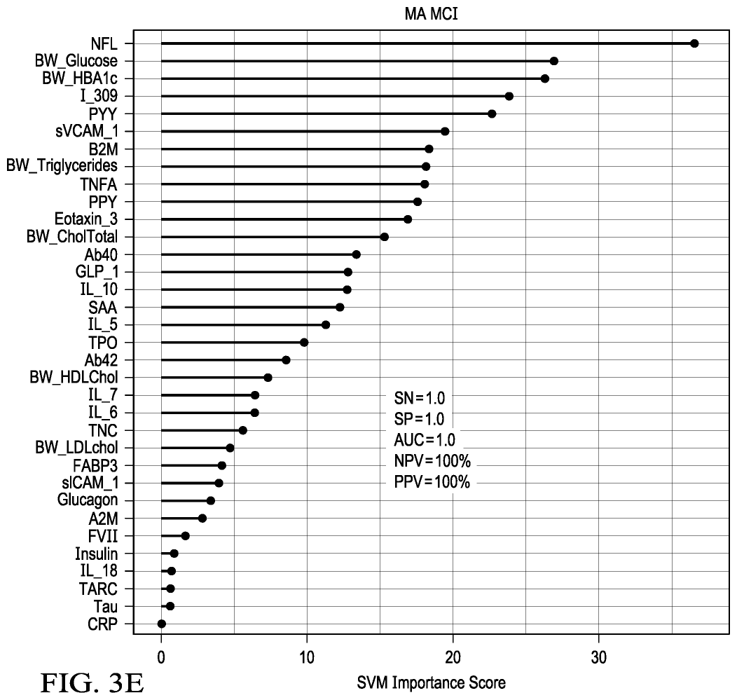

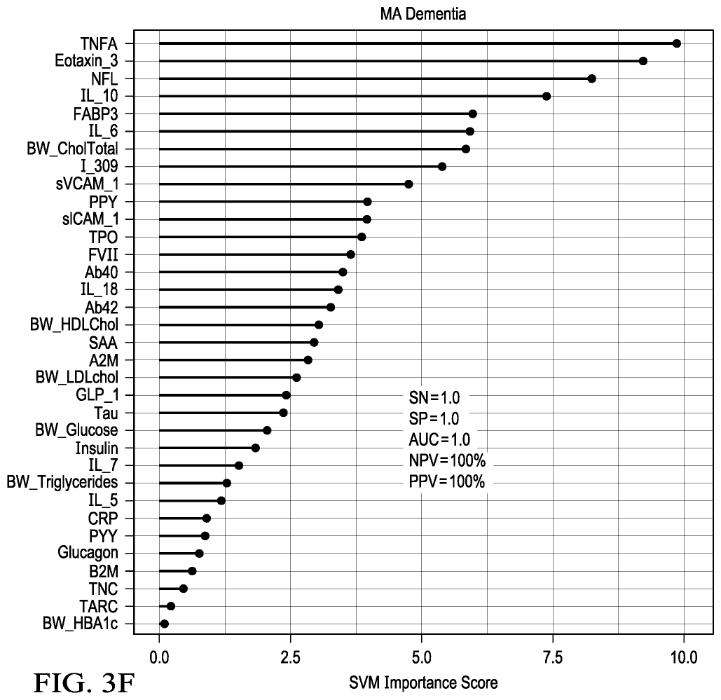

Blood Profile of N+ Split By Ethnicity and Diagnosis. Next, the inventors examined the blood-based profile for detecting N+ split by ethnic group as well as diagnostic status (FIG. 3). Non-Hispanic Whites: Controls (FIG. 3A)—AUC=0.99, SN=0.97, SP=0.97, plasma NfL AUC=0.62; MCI (FIG. 3B)—AUC=1.0, SN=1.0, SP=1.0, plasma NfL AUC=0.62; dementia (FIG. 3C)—AUC=1.0, SN=1.0, SP=1.0, plasma NfL AUC=0.67. Mexican Americans: Controls (FIG. 3D)—AUC=1.0, SN=1.0, SP=1.0, plasma NfL AUC=0.65; MCI (FIG. 3E)— AUC=1.0, SN=1.0, SP=1.0, plasma NfL AUC=0.74; dementia (FIG. 3F)—AUC=1.0, SN=1.0, SP=1.0, plasma NfL AUC=0.57. Again, the relative importance of the blood biomarkers varied substantially by both ethnic status and disease severity.

Thus, the present invention is a blood-based profile for detecting underlying neurodegeneration (as measured by MRI). The overall profile was highly significant across ethnic groups and diagnostic classifications. However, it is important to note that these profiles not only changed by ethnicity but also by disease duration. Given that N is a non-specific marker of brain damage, it is also highly likely that the underlying factors leading to neurodegeneration are just as non-specific and multi-factorial. Therefore, leveraging that heterogeneity may very well provide an optimal setting for targeted, precision medicine-based interventions. The current data show that: (1) blood-based profiles can be highly accurate in detecting underlying neurodegeneration and (2) targeted, disease-severity driven, precision medicine approaches may improve therapeutic outcomes.

Thus, the present invention includes the treatment of patients based on the blood-based profiles. The application of these medications (and others) for targeted, multi-modal neuroprotective prevention is summarized in Table 2. From a prevention trial pipeline standpoint with view on A, T and N. First, most community-dwelling older adults ages 50-70 will be amyloid and tau negative. It is likely that prevention efforts will be most successful if implemented as early as possible. N+ rates were 30% among cognitively older adults ages 50 and above; however, that prevalence rate declines to 16% if restricted to the age range above. When looking at FIGS. 3A and 3D, the blood profile of neurodegeneration is multi-factorial, as expected, with markers relevant to diabetes, heart disease and inflammation all playing prominent roles. Therefore, a preventative neuroprotection strategy can use currently available medications based on the specific individual clinical labs. In Table 2, multiple examples are provided. Person 1 is a 70-year-old non-Hispanic white patient who is amyloid positive that has elevated clinical labs related to cardiovascular disease. Cardiovascular disease markers were strong predictors of neurodegeneration specifically among non-Hispanics. Therefore, in Person 1, an anti-amyloid agent would be the primary (initial) intervention with the second (and/or 3rd) multi-modal level interventions targeting the clinical lab defined abnormalities. Person 2, however, is 50-year-old Mexican Americans who only has diabetes. The inventors found that duration of diabetes was a powerful driver of N among Mexican Americans. Therefore, in Person 2, the person-centered neuroprotection prevention strategy is to target and control the diabetes. Numerous other multi-modal opportunities are readily apparent for targeting neuroprotection as a prevention strategy in Table 2.

TABLE 2
Person-centered risk factors for prevention among N+ cognitively normal
older adults

| | Potential Therapeutic | | |
|---|---|---|---|
| Therapeutic Target | Approach | Person 1 | Person 2 |
| Amyloid Positive | Amyloid lowering agent | X¹ | |
| Tau Positive | Tau lowering agent | | |
| HDL/LDL/Triglycerides | Cholesterol medications | X² | |
| Glucose, HbAlc | Diabetes Medications | | X¹ |

| | |
|---|---|
| Inflammation | Anti-inflammatory |
| | Medications |

NOTE:
X¹ = primary/initial therapeutic;
X² = secondary multi-modal therapeutic

Given the recent literature strongly supporting plasma NfL as a putative biomarker of neurodegeneration, a viable question is if this single marker can serve the purpose of the COU proposed here. However, the inventors recently completed a large-scale examination of the clinical parameters of plasma NfL among n=1,625 participants of the HABLE study. It was found that plasma NfL was significantly associated with neuropsychological test scores and global amyloid SUVR. NfL levels varied by diagnostic group. However, NfL was significantly impacted by demographic factors and medical comorbidities, which likely contributed to the lack of utility of plasma NfL as a diagnostic biomarker for MCI or dementia. Therefore, plasma NfL was directly compared to the profile approach. There are multiple advantage to a single marker; however, plasma NfL did not achieve acceptable AUCs. This is likely due to the substantial impact of medical comorbidities and demographic factors on plasma NfL levels that causes additional “spread” in values on top of the impact of neurodegeneration causing cognitive loss.

According to 2019 Census Bureau data [27], Hispanics make up the largest minority population in the U.S. In fact, approximately 50% of the U.S. population growth from 2010-2019 was due to an increase in the Hispanic population [28]. The percentage of Hispanics aged 65 and older will triple by the year 2050[29] and this ethnic group will experience the largest increase in AD and AD related dementia (ADRD) diagnoses among any racial/ethnic group by 2060[30]. Approximately 65% of Hispanics in the U.S. are of Mexican American ethnicity [31]; however, few studies have explicitly examined MCI and AD among Mexican Americans. Therefore, it is important to consider the factors contributing to A, T and N across diverse groups to have a comprehensive picture and to build more powerful intervention opportunities and strategies.

These results show the utility of a blood-based screening tool for neurodegeneration. Additionally, these findings show additional targets for novel prevention and intervention trials along with specific multi-modal therapeutic options for these neuroprotection trials.

In one embodiment, the present invention includes a method for detecting neurodegeneration and treating a subject that is of Mexican American or non-Hispanic white origin, the method comprising, consisting essentially of, or consisting of: obtaining a blood, plasma or serum sample from the subject comprising both biochemical and protein biomarkers; determining if the subject is of Mexican American or of non-Hispanic white origin; measuring in the blood, plasma or serum sample a level of one or more biochemical biomarkers selected from the group consisting of: cholesterol, triglycerides, HDL cholesterol, glucose, HBAc1, LDL cholesterol, and glucagon; or measuring in the blood, plasma or serum sample an expression level of one or more protein biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10, or both biochemical biomarkers and protein biomarkers; comparing the level of expression from the sample with a statistical sample representative of the subject of Mexican American or of non-Hispanic white origin, suspected of having neurodegeneration; and treating the subject with a treatment that targets neurodegeneration or neuronal injury, wherein the neurodegeneration or neuronal injury is measured by [18F]-fluorodeoxyglucose-PET, structural MRI, or CSF total tau. In one aspect, the subject is of Mexican American origin and suffering neurodegeneration with or without mild cognitive impairment or dementia. In another aspect, the subject is of Mexican American origin, neurodegeneration is screened for by detecting the level of biochemical biomarkers and the level of expression of biomarkers, selected from NFL, A2M, Ab40, B2M, FABP3, HBA1c, HDL cholesterol, IL-6, Ab42, IL-5, TNC, eotaxin-3, PPY, tau, glucose, triglycerides, SAA, TNFα, cholesterol, IL-7, TPO, sICAM-1, IL-18, Factor VII, sVCAM-1, LDL cholesterol, CRP, GLP-1, insulin, IL-10, TARC, PYY, glucagon, and I309, and optionally, wherein the biochemical and protein biomarkers are in descending order. In another aspect, if the subject is of Mexican American origin and suffering from mild cognitive impairment, neurodegeneration is screened for by detecting the level of biochemical biomarkers and the level of expression of biomarkers, selected from NFL, glucose, HBAc1, 1309, PYY, sVCAM-1, B2M, triglycerides, TNFα, PPY, eotaxin-3, cholesterol, Ab40, GLP-1, IL-10, SAA, IL-5, and TPO, and optionally, wherein the biochemical and protein biomarkers are in descending order. In another aspect, if the subject is of Mexican American origin and suffering from dementia, neurodegeneration is screened for by detecting the level of biochemical biomarkers and the level of expression of biomarkers, selected from TNFα, eotaxin-3, NFL, IL-10, FABP3, IL-6, cholesterol, I309, sVCAM-1, PPY, sICAM-1, TPO, factor VII, Ab40, IL-18, Ab42, HDL cholesterol, SAA, A2M, LDL cholesterol, and GLP-1, and optionally, wherein the biochemical and protein biomarkers are in descending order. In another aspect, the subject is of non-Hispanic white origin and suffering from neurodegeneration with or without mild cognitive impairment or dementia. In another aspect, if the subject is of non-Hispanic white origin neurodegeneration is screened for by detecting the level of biochemical biomarkers and the level of expression of biomarkers, selected from cholesterol, A2M, Factor VII, TNC, HDL cholesterol, IL-18, Ab42, NFL, Ab40, LDL cholesterol, TARC, IL-6, TNFα, B2M, IL-5, TPO, I309, triglycerides, SAA, insulin, PPY, tau, GLP-1, glucose, FABP3, sICAM-1, sVCAM-1, PYY, eotaxin-3, IL-7, CRP, IL-10, HBA1c, and glucagon, and optionally, wherein the biochemical and protein biomarkers are in descending order. In another aspect, if the subject is of non-Hispanic white origin and suffering from mild cognitive impairment, neurodegeneration is screened for by detecting the level of biochemical biomarkers and the level of expression of biomarkers, selected from triglycerides, HDL cholesterol, glucose, HBAc1, A2M, GLP-1, IL-7, IL-6, PYY, IL-10, glucagon, Ab40, FABP3, Ab42, eotaxin-3, and Factor VII, and optionally, wherein the biochemical and protein biomarkers are in descending order. In another aspect, if the subject is of non-Hispanic white origin and suffering from dementia, neurode generation is screened for by detecting the level of biochemical biomarkers and the level of expression of biomarkers, selected from HDL cholesterol, Ab42, A2M, FABP3, TNC, TARC, glucose, TPO, CRP, and eotaxin-3, and optionally, wherein the biochemical and protein biomarkers are in descending order. In another aspect, the method further comprises wherein: (i) when the expression level of the one or more biomarkers in the blood, plasma or serum sample is statistically similar to the average expression level of the corresponding one or more biomarkers obtained from a group of individuals in the statistical sample who do not have neurodegeneration, the subject is excluded from further testing for neurodegeneration; or (ii) when the expression level of the one or more biomarkers in the blood, plasma or serum sample is not statistically similar to the average expression level of the corresponding one or more biomarkers obtained from a group of individuals in the statistical sample who have been diagnosed with neurodegeneration, the subject is further tested for neurodegeneration. In another aspect, the method further testing for neurodegeneration excluded is selected from MRI, PET, or spinal fluid tap. In another aspect, the expression level of the one or more biomarkers in the blood, plasma or serum sample is statistically similar to the average expression level of the corresponding one or more biomarkers obtained from a group of individuals in the statistical sample who have been diagnosed with neurodegeneration, the method further comprises referring the subject to a specialist for diagnostic testing for a neurological disease or disorder.

In another embodiment, the present invention includes a method of screening a subject for exclusion from a clinical trial for a neuroprotection agent, the method comprising, consisting essentially of, or consisting of: obtaining a blood, plasma or serum sample from the subject comprising both biochemical and protein biomarkers; determining if the subject is of Mexican American or of non-Hispanic white origin; measuring in the blood, plasma or serum sample a level of one or more biochemical biomarkers selected from the group consisting of: cholesterol, triglycerides, HDL cholesterol, glucose, HBAc1, LDL cholesterol, and glucagon; or measuring in the blood, plasma or serum sample an expression level of one or more protein biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10, or both biochemical and protein biomarkers; comparing the level of expression from the sample with a statistical sample representative of the subject of Mexican American or of non-Hispanic white origin, suspected of having neurodegeneration; and determining that the subject has a neurodegeneration profile based on the expression level of the one or more biomarkers; and excluding the subject for inclusion in the clinical trial targeting neuroprotection if the subject has the neurodegeneration profile. In one aspect, if the subject is of Mexican American origin, the neurodegeneration profile is screened for by detecting the level of biochemical biomarkers and the level of expression of biomarkers, selected from: NFL, A2M, Ab40, B2M, FABP3, HBA1c, HDL cholesterol, IL-6, Ab42, IL-5, TNC, eotaxin-3, PPY, tau, glucose, triglycerides, SAA, TNFα, cholesterol, IL-7, TPO, sICAM-1, IL-18, Factor VII, sVCAM-1, LDL cholesterol, CRP, GLP-1, insulin, IL-10, TARC, PYY, glucagon, and I309, and optionally, wherein the biochemical and protein biomarkers are in descending order. In another aspect, if the subject is of Mexican American origin and suffering from mild cognitive impairment, the neurodegeneration profile is screened for by detecting the level of biochemical biomarkers and the level of expression of biomarkers, selected from NFL, glucose, HBAc1, 1309, PYY, sVCAM-1, B2M, triglycerides, TNFα, eotaxin-3, cholesterol, Ab40, GLP-1, IL-10, SAA, IL-5, and TPO, and optionally, wherein the biochemical and protein biomarkers are in descending order. In another aspect, if the subject is of Mexican American origin and suffering from dementia, the neurodegeneration profile is screened for by detecting the level of biochemical biomarkers and the level of expression of biomarkers, selected from TNFα, eotaxin-3, NFL, IL-10, FABP3, IL-6, cholesterol, I309, sVCAM-1, PPY, sICAM-1, TPO, factor VII, Ab40, IL-18, Ab42, HDL cholesterol, SAA, A2M, LDL cholesterol, and GLP-1, and optionally, wherein the biochemical and protein biomarkers are in descending order. In another aspect, if the subject is of non-Hispanic white origin the neurodegeneration profile is screened for by detecting the level of biochemical biomarkers and the level of expression of biomarkers, selected from cholesterol, A2M, Factor VII, TNC, HDL cholesterol, IL-18, Ab42, NFL, Ab40, LDL cholesterol, TARC, IL-6, TNFα, B2M, IL-5, TPO, I309, triglycerides, SAA, insulin, PPY, tau, GLP-1, glucose, FABP3, sICAM-1, sVCAM-1, PYY, eotaxin-3, IL-7, CRP, IL-10, HBA1c, and glucagon, and optionally, wherein the biochemical and protein biomarkers are in descending order. In another aspect, if the subject is of non-Hispanic white origin and suffering from mild cognitive impairment, the neurodegeneration profile is screened for by detecting the level of biochemical biomarkers and the level of expression of biomarkers, selected from triglycerides, HDL cholesterol, glucose, HBAc1, A2M, GLP-1, IL-7, IL-6, PYY, IL-10, glucagon, Ab40, FABP3, Ab42, eotaxin-3, and Factor VII, and optionally, wherein the biochemical and protein biomarkers are in descending order. In another aspect, if the subject is of non-Hispanic white origin and suffering from dementia, the neurodegeneration profile is screened for by detecting the level of biochemical biomarkers and the level of expression of biomarkers, selected from HDL cholesterol, Ab42, A2M, FABP3, TNC, TARC, glucose, TPO, CRP, and eotaxin-3, and optionally, wherein the biochemical and protein biomarkers are in descending order.

In another embodiment, the present invention includes a method for detecting neurodegeneration in a subject that is of Mexican American origin, the method comprising, consisting essentially of, or consisting of: (a) obtaining or having obtained a blood, plasma or serum sample from the subject comprising both biochemical and protein biomarkers; (b) determining if the subject has diabetes and a duration of the diabetes; (c) measuring in the blood, plasma or serum sample one or more biochemical biomarkers of diabetes selected from HbA1c, glucose; or (d) measuring in the blood, plasma or serum sample an expression level of one or more biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10; (e) comparing the level of expression from the sample with a statistical sample representative of the subject of Mexican American or of non-Hispanic white origin, suspected of having a neurodegenerative disease; and (f) treating the subject with a treatment that targets neurodegeneration or neuronal injury, wherein the neurodegeneration or neuronal injury is measured by [18F]-fluorodeoxyglucose-PET, structural MRI, or CSF total tau. In another aspect, the method further comprises at least one of: determining if the subject has neurodegeneration by detecting the level of expression of biomarkers, selected from NFL, A2M, Ab40, B2M, FABP3, IL-6, Ab42, IL-5, TNC, eotaxin-3, PYY, PPY, tau, SAA, TNFα, cholesterol, IL-7, TPO, sICAM-1, IL-18, Factor VII, sVCAM-1, CRP, GLP-1, insulin, IL-10, TARC, and I309, and optionally, wherein the biomarkers are in descending order;

In another embodiment, the present invention includes a method for detecting neurodegeneration in a subject of non-Hispanic white origin, the method comprising: (a) obtaining a blood, plasma or serum sample from the subject comprising both biochemical and protein biomarkers; (b) determining if the subject has a cardiovascular disease and hypertension; (c) measuring in the blood, plasma or serum sample a level of one or more biochemical biomarkers selected from LDL, and total cholesterol; or (d) measuring in the blood, plasma or serum sample an expression level of one or more biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10; (e) comparing the level of expression from the sample with a statistical sample representative of the subject of Mexican American or of non-Hispanic white origin, suspected of having a neurodegenerative disease; and (f) treating the subject with a treatment that targets neurodegeneration or neuronal injury, wherein the neurodegeneration or neuronal injury is measured by [18F]-fluorodeoxyglucose-PET, structural MRI, or CSF total tau. In one aspect, the method further comprises determining if the subject has a neurodegenerative disease by determining the level of expression of biomarkers selected from A2M, Factor VII, TNC, IL-18, Ab42, NFL, Ab40, TARC, IL-6, TNFα, B2M, IL-5, TPO, I309, triglycerides, SAA, insulin, PYY, PPY, tau, GLP-1, FABP3, sICAM-1, sVCAM-1, eotaxin-3, IL-7, CRP, and IL-10, and optionally, wherein the biomarkers are in descending order.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

[1] Jack Jr. C R, Bennett D A, Blennow K, Carrillo M C, Dunn B, Haeberlein S B, et al. NIA-AA Research Framework: Toward a biological definition of Alzheimer's disease. Alzheimers Dement 2018; 14:535-62. doi.org/10.1016/j.jalz.2018.02.018.
[2] Pan D, Zeng A, Jia L, Huang Y, Frizzell T, Song X. Early Detection of Alzheimer's Disease Using Magnetic Resonance Imaging: A Novel Approach Combining Convolutional Neural Networks and Ensemble Learning. Front Neurosci 2020; 14. doi.org/10.3389/fnins 2020.00259.
[3] Aderghal K, Afdel K, Benois-Pineau J, Catheline G. Improving Alzheimer's stage categorization with Convolutional Neural Network using transfer learning and different magnetic resonance imaging modalities. Heliyon 2020; 6. doi.org/10.1016/j.heliyon.2020.e05652.
[4] Jack C R, Wiste H J, Therneau T M, Weigand S D, Knopman D S, Mielke M M, et al. Associations of Amyloid, Tau, and Neurodegeneration Biomarker Profiles with Rates of Memory Decline among Individuals Without Dementia. JAMA—J Am Med Assoc 2019; 321:2316-25. doi.org/10.1001/jama.2019.7437.
[5] Seo E H, Park W Y, Choo I H. Structural MRI and amyloid PET imaging for prediction of conversion to Alzheimer's disease in patients with mild cognitive impairment: A meta-analysis. Psychiatry Investig 2017; 14:205-15. doi.org/10.4306/pi.2017.14.2.205.
[6] Zeng Q, Li K, Luo X, Wang S, Xu X, Li Z, et al. Distinct Atrophy Pattern of Hippocampal Subfields in Patients with Progressive and Stable Mild Cognitive Impairment: A Longitudinal MRI Study. J Alzheimer's Dis 2020:1-11. doi.org/10.3233/jad-200775.
[7] Search of: neuroprotection—List Results—ClinicalTrials.gov n.d. clinicaltrials.gov/ct2/results?cond=neuroprotection&term=& cntry=& state=&city=& dist= (accessed Dec. 29, 2020).
[8] AD+ADRD Research Implementation Milestone Database National Institute on Aging n.d. www.nia.nih.gov/research/milestones (accessed Dec. 27, 2020).
[9] O'Bryant S E, Xiao G, Edwards M, Devous M, Gupta V B, Martins R, et al. Biomarkers of Alzheimer's disease among Mexican Americans. J Alzheimer's Dis 2013. doi.org/10.3233/JAD-122074.
[10] O'Bryant S E, Johnson L, Reisch J, Edwards M, Hall J, Barber R, et al. Risk factors for mild cognitive impairment among Mexican Americans. Alzheimer's Dement 2013. doi.org/10.1016/j.jalz.2012.12.007.
[11] O'Bryant S E, Zhang F, Johnson L A, Hall J, Edwards M, Grammas P, et al. A Precision Medicine Model for Targeted NSAID Therapy in Alzheimer's Disease. J Alzheimers Dis 2018; 66:97-104. doi.org/10.3233/JAD-180619.
[12] Folstein M F, Folstein S E, McHugh P R. “Mini-mental state”. A practical method for grading the cognitive state of patients for the clinician. J Psychiatr Res 1975; 12:189-98.
[13] Weschler D. Weschler memory scale—revised. San Antonio: Psychological Corporation; 1987.
[14] Reitan R M, Wolfson D. The Halstead-Reitan Neuropsychological Test Battery: Theory and Interpretation. Tucson, AZ: Neuropsychology Press; 1985.
[15] Gonzälez H M, Mungas D, Haan M N. A verbal learning and memory test for English- and Spanish-speaking older Mexican-American adults. Clin Neuropsychol 2002. doi.org/10.1076/clin.16.4.439.13908.
[16] Lezak M D, Howieson D B, Loring D W. Neuropsychological Assessment. 4th ed. Oxford: Oxford University Press; 2004.
[17] O'Bryant S E, Edwards M, Johnson L, Hall J, Gamboa A, O′jile J. Texas Mexican American adult normative studies: Normative data for commonly used clinical neuropsychological measures for English- and Spanish-speakers. Dev Neuropsychol 2018. doi.org/10.1080/87565641.2017.1401628.
[18F] Sierra Sanjurjo N, Montaties P, Sierra Matamoros F A, Burin D. Estimating Intelligence in Spanish: Regression Equations with the Word Accentuation Test and Demographic Variables in Latin America. Appl Neuropsychol 2015. doi.org/10.1080/23279095.2014.918543.
[19] Berg L. Clinical Dementia Rating (CDR). Psychopharmacol Bull 1988.
[20] O'Bryant S E, Gupta V, Henriksen K, Edwards M, Jeromin A, Lista S, et al. Guidelines for the standardization of preanalytic variables for blood-based biomarker studies in Alzheimer's disease research. Alzheimer's Dement 2015. doi.org/10.1016/j jalz.2014.08.099.
[21] O'Bryant S E, Edwards M, Johnson L, Hall J, Villarreal A E, Britton G B, et al. A blood screening test for Alzheimer's disease. Alzheimer's Dement Diagnosis, Assess Dis Monit 2016. doi.org/10.1016/j.dadm.2016.06.004.
[22] O'Bryant S E, Ferman T J, Zhang F, Hall J, Pedraza O, Wszolek Z K, et al. A proteomic signature for dementia with Lewy bodies. Alzheimer's Dement Diagnosis, Assess Dis Monit 2019. doi.org/10.1016/j.dadm.2019.01.006.
[23] O'Bryant S E, Edwards M, Zhang F, Johnson L A, Hall J, Kuras Y, et al. Potential two-step proteomic signature for Parkinson's disease: Pilot analysis in the Harvard Biomarkers Study. Alzheimer's Dement Diagnosis, Assess Dis Monit 2019. doi.org/10.1016/j.dadm.2019.03.001.
[24] Petersen M, Zhang F, Krinsky-McHale S J, Silverman W, Lee J H, Pang D, et al. Proteomic profiles of prevalent mild cognitive impairment and Alzheimer's disease among adults with Down syndrome. Alzheimer's Dement Diagnosis, Assess Dis Monit (Special Collect Biomarkers Alzheimer's Dis among Adults with Down Syndr n.d.
[25] R_Development_Core_Team. R: A language and environment for statistical computing 2009. www.r-project.org.
[26] LaValle S M, Branicky M S. On the relationship between classical grid search and probabilistic roadmaps. Springer Tracts Adv. Robot., 2004. doi.org/10.1007/978-3-540-45058-O_5.
[27] U.S. Census Bureau QuickFacts: United States n.d. https://www.census.gov/quickfacts/fact/table/US/PST045219 (accessed Dec. 8, 2020).
[28] US Hispanic population reached new high in 2019, but growth slowed Pew Research Center n.d. www.pewresearch.org/fact-tank/2020/07/07/u-s-hispanic-population-surpassed-60-million-in-2019-but-growth-has-slowed/(accessed Dec. 8, 2020).
[29] Linda A. Jacobsen et al. America's Aging Population. Popul Bull 2011; 66.
[30] Matthews K A, Xu W, Gaglioti A H, Holt J B, Croft J B, Mack D, et al. Racial and ethnic estimates of Alzheimer's disease and related dementias in the United States (2015-2060) in adults aged ≥65 years. Alzheimer's Dement 2019. doi.org/10.1016/j.jalz.2018.06.3063.
[31] CensusBureau US. American Fact Finder 2004; 2006. www.census.gov/.

What is claimed is:

1. A method for detecting neurodegeneration and treating a subject that is of Mexican American or non-Hispanic white origin, the method comprising:
obtaining a blood, plasma or serum sample from the subject comprising both biochemical and protein biomarkers;
determining if the subject is of Mexican American or of non-Hispanic white origin;
measuring in the blood, plasma or serum sample a level of one or more biochemical biomarkers selected from the group consisting of: cholesterol, triglycerides, HDL cholesterol, glucose, HBAc1, LDL cholesterol, and glucagon; or
measuring in the blood, plasma or serum sample an expression level of one or more protein biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10, or both biochemical biomarkers and protein biomarkers;
comparing the level of expression from the sample with a statistical sample representative of the subject of Mexican American or of non-Hispanic white origin, suspected of having neurodegeneration; and
treating the subject with a treatment that targets neurodegeneration or neuronal injury, wherein the neurodegeneration or neuronal injury is measured by [18F]-fluorodeoxyglucose-PET, structural MRI, or CSF total tau.

2. The method of claim 1, wherein the subject is of Mexican American origin and suffering neurodegeneration with or without mild cognitive impairment or dementia.

3. The method of claim 1, wherein if the subject is of Mexican American origin, neurodegeneration is screened for by detecting the level of biochemical biomarkers and the level of expression of biomarkers, selected from NFL, A2M, Ab40, B2M, FABP3, HBA1c, HDL cholesterol, IL-6, Ab42, IL-5, TNC, eotaxin-3, PPY, tau, glucose, triglycerides, SAA, TNFα, cholesterol, IL-7, TPO, sICAM-1, IL-18, Factor VII, sVCAM-1, LDL cholesterol, CRP, GLP□1, insulin, IL-10, TARC, PYY, glucagon, and I309, and optionally, wherein the biochemical and protein biomarkers are in descending order.

4. The method of claim 1, wherein if the subject is of Mexican American origin and suffering from mild cognitive impairment, neurodegeneration is screened for by detecting the level of biochemical biomarkers and the level of expression of biomarkers, selected from NFL, glucose, HBAc1, 1309, PYY, sVCAM-1, B2M, triglycerides, TNFα, PPY, eotaxin-3, cholesterol, Ab40, GLP-1, IL-10, SAA, IL-5, and TPO, and optionally, wherein the biochemical and protein biomarkers are in descending order.

5. The method of claim 1, wherein if the subject is of Mexican American origin and suffering from dementia, neurodegeneration is screened for by detecting the level of biochemical biomarkers and the level of expression of biomarkers, selected from TNFα, eotaxin-3, NFL, IL-10, FABP3, IL-6, cholesterol, I309, sVCAM-1, PPY, sICAM-1, TPO, factor VII, Ab40, IL-18, Ab42, HDL cholesterol, SAA, A2M, LDL cholesterol, and GLP-1, and optionally, wherein the biochemical and protein biomarkers are in descending order.

6. The method of claim 1, wherein the subject is of non-Hispanic white origin and suffering from neurodegeneration with or without mild cognitive impairment or dementia.

7. The method of claim 1, wherein if the subject is of non-Hispanic white origin neurodegeneration is screened for by detecting the level of biochemical biomarkers and the level of expression of biomarkers, selected from cholesterol, A2M, Factor VII, TNC, HDL cholesterol, IL-18, Ab42, NFL, Ab40, LDL cholesterol, TARC, IL-6, TNFα, B2M, IL-5, TPO, I309, triglycerides, SAA, insulin, PPY, tau, GLP-1, glucose, FABP3, sICAM-1, sVCAM-1, PYY, eotaxin-3, IL-7, CRP, IL-10, HBA1c, and glucagon, and optionally, wherein the biochemical and protein biomarkers are in descending order.

8. The method of claim 1, wherein if the subject is of non-Hispanic white origin and suffering from mild cognitive impairment, neurodegeneration is screened for by detecting the level of biochemical biomarkers and the level of expression of biomarkers, selected from triglycerides, HDL cholesterol, glucose, HBAc1, A2M, GLP-1, IL-7, IL-6, PYY, IL-10, glucagon, Ab40, FABP3, Ab42, eotaxin-3, and Factor VII, and optionally, wherein the biochemical and protein biomarkers are in descending order.

9. The method of claim 1, wherein if the subject is of non-Hispanic white origin and suffering from dementia, neurodegeneration is screened for by detecting the level of biochemical biomarkers and the level of expression of biomarkers, selected from HDL cholesterol, Ab42, A2M, FABP3, TNC, TARC, glucose, TPO, CRP, and eotaxin-3, and optionally, wherein the biochemical and protein biomarkers are in descending order.

10. The method of claim 1, wherein:
(i) when the expression level of the one or more biomarkers in the blood, plasma or serum sample is statistically similar to an average expression level of a corresponding one or more biomarkers obtained from a group of individuals in the statistical sample who do not have neurodegeneration, the subject is excluded from further testing for neurodegeneration; or
(ii) when the expression level of the one or more biomarkers in the blood, plasma or serum sample is not statistically similar to the average expression level of the corresponding one or more biomarkers obtained from a group of individuals in the statistical sample who have been diagnosed with neurodegeneration, the subject is further tested for neurodegeneration.

11. The method of claim 10, wherein the further testing for neurodegeneration excluded is selected from MRI, PET, or spinal fluid tap.

12. The method of claim 10, wherein when the expression level of the one or more biomarkers in the blood, plasma or serum sample is statistically similar to the average expression level of the corresponding one or more biomarkers obtained from a group of individuals in the statistical sample who have been diagnosed with neurodegeneration, the method further comprises referring the subject to a specialist for diagnostic testing for a neurological disease or disorder.

13. A method of screening a subject for exclusion from a clinical trial for a neuroprotection agent, the method comprising:
obtaining a blood, plasma or serum sample from the subject comprising both biochemical and protein biomarkers;
determining if the subject is of Mexican American or of non-Hispanic white origin;
measuring in the blood, plasma or serum sample a level of one or more biochemical biomarkers selected from the group consisting of: cholesterol, triglycerides, HDL cholesterol, glucose, HBAc1, LDL cholesterol, and glucagon; or
measuring in the blood, plasma or serum sample an expression level of one or more protein biomarkers selected from the group consisting of: alpha-2-microglobulin (A2M), Factor VII, tenacin C (TNC), IL-18, Ab42, neurofilament light (NfL), Aβ40, Chemokine (C-C Motif) Ligand 17 (TARC), IL-6, tumor necrosis factor alpha (TNFα), beta-2-microglobulin (B2M), IL-5, thrombopoietin (TPO), I309, serum amyloid A1 cluster (SAA), insulin, pancreatic polypeptide (PPY), peptide YY (PYY), tau, GLP-1, fatty acid binding protein 3 (FABP3), soluble intracellular adhesion molecule (sICAM-1), soluble vascular adhesion molecule (sVCAM-1), eotaxin-3, IL-7, C-reactive protein (CRP), and IL-10, or both biochemical and protein biomarkers;
comparing the level of expression from the sample with a statistical sample representative of a subject of Mexican American or of non-Hispanic white origin, suspected of having neurodegeneration; and
determining that the subject has a neurodegeneration profile based on the expression level of the one or more biomarkers; and
excluding the subject for inclusion in the clinical trial targeting neuroprotection if the subject has the neurodegeneration profile.

14. The method of claim 13, wherein if the subject is of Mexican American origin, the neurodegeneration profile is screened for by detecting the level of biochemical biomarkers and the level of expression of biomarkers, selected from: NFL, A2M, Ab40, B2M, FABP3, HBA1c, HDL cholesterol, IL-6, Ab42, IL-5, TNC, eotaxin-3, PPY, tau, glucose, triglycerides, SAA, TNFα, cholesterol, IL-7, TPO, sICAM-1, IL-18, Factor VII, sVCAM-1, LDL cholesterol, CRP, GLP-1, insulin, IL-10, TARC, PYY, glucagon, and I309, and optionally, wherein the biochemical and protein biomarkers are in descending order.

15. The method of claim 13, wherein if the subject is of Mexican American origin and suffering from mild cognitive impairment, the neurodegeneration profile is screened for by detecting the level of biochemical biomarkers and the level of expression of biomarkers, selected from NFL, glucose, HBAc1, 1309, PYY, sVCAM-1, B2M, triglycerides, TNFα, eotaxin-3, cholesterol, Ab40, GLP-1, IL-10, SAA, IL-5, and TPO, and optionally, wherein the biochemical and protein biomarkers are in descending order.

16. The method of claim 13, wherein if the subject is of Mexican American origin and suffering from dementia, the neurodegeneration profile is screened for by detecting the level of biochemical biomarkers and the level of expression of biomarkers, selected from TNFα, eotaxin-3, NFL, IL-10, FABP3, IL-6, cholesterol, I309, sVCAM-1, PPY, sICAM-1, TPO, factor VII, Ab40, IL-18, Ab42, HDL cholesterol, SAA, A2M, LDL cholesterol, and GLP-1, and optionally, wherein the biochemical and protein biomarkers are in descending order.

17. The method of claim 13, wherein if the subject is of non-Hispanic white origin the neurodegeneration profile is screened for by detecting the level of biochemical biomarkers and the level of expression of biomarkers, selected from cholesterol, A2M, Factor VII, TNC, HDL cholesterol, IL-18, Ab42, NFL, Ab40, LDL cholesterol, TARC, IL-6, TNFα, B2M, IL-5, TPO, I309, triglycerides, SAA, insulin, PPY, tau, GLP-1, glucose, FABP3, sICAM-1, sVCAM-1, PYY, eotaxin-3, IL-7, CRP, IL-10, HBA1c, and glucagon, and optionally, wherein the biochemical and protein biomarkers are in descending order.

18. The method of claim 13, wherein if the subject is of non-Hispanic white origin and suffering from mild cognitive impairment, the neurodegeneration profile is screened for by detecting the level of biochemical biomarkers and the level of expression of biomarkers, selected from triglycerides, HDL cholesterol, glucose, HBAc1, A2M, GLP-1, IL-7, IL-6, PYY, IL-10, glucagon, Ab40, FABP3, Ab42, eotaxin-3, and Factor VII, and optionally, wherein the biochemical and protein biomarkers are in descending order.

19. The method of claim 13, wherein if the subject is of non-Hispanic white origin and suffering from dementia, the neurodegeneration profile is screened for by detecting the level of biochemical biomarkers and the level of expression of biomarkers, selected from HDL cholesterol, Ab42, A2M, FABP3, TNC, TARC, glucose, TPO, CRP, and eotaxin-3, and optionally, wherein the biochemical and protein biomarkers are in descending order.
